(19) United States
(12) Patent Application Publication
(10) Pub. No.: US 2012/0232525 A1
(43) Pub. Date: Sep. 13, 2012

(54) SUCTION DEVICE

(76) Inventor: Ashok Kumar Golagani, Northolt (GB)

(21) Appl. No.: 13/392,859

(22) PCT Filed: Aug. 18, 2010
(86) PCT No.: PCT/GB2010/051362

§ 371 Date: May 21, 2012

(30) Foreign Application Priority Data
Aug. 28, 2009 (GB) 0915034.3

(51) Int. Cl. A61M 1/00; A61M 31/00

(52) U.S. Cl. 604/514; 604/540

(57) ABSTRACT

There is provided a device comprising a funnel connected to a suction tube connectable to a suction device, the funnel comprising a reversibly inflatable portion. The device is preferably a nasopharangeal device suitable for insertion through a mouth of a patient, the funnel being formed so as to be sealably locatable in or around the nasopharangeal opening of the patient. There are further provided methods for protecting an airway of a patient from nasal-derived liquid and/or applying medicament and/or packing to a nasopharynx, oropharynx or nasal cavity of a patient using the inventive device. The device provides a system in which misting of endoscopic lenses is reduced or prevented. Embodiments of the device allow for the strength of applied suction to be controlled.

### FIELD OF INVENTION

The invention relates to a device for use in nasal surgery, to prevent blood and other fluids draining into a patient's airway. The device also enables the application of medicaments and/or packing materials, which can be held in an appropriate position by use of the device.

### BACKGROUND

During nose operations a clear operative space is required and it is very difficult to clear bleeding from the nose given the limited space. To protect the airway from blood during nose operations, anaesthetists typically put packing in the throat. This is time consuming and there is a risk of trauma to the lining of throat, as well as problems associated with managing the airway and the risk of a failure to fully remove the packing at the end of the procedure. Furthermore, packing of the throat has the result that blood in the nasal cavity must be cleared from the operative side, causing breaks in the surgeon's work, increasing the time taken for the procedure to be completed and causing trauma to the lining of the nose. In addition, this does not provide safe and continuous suction clearance of blood during operations.

Pre-operative nasal sprays and medicaments may also be used to minimise bleeding during an operation. However, most of these medications drop down to the nasopharynx within a few seconds, making these medications ineffective. There is no device to hold these medications in place for a desired period of time.

In addition to the problems outlined above, with the use of nasal endoscopes another problem is misting of the endoscopic lenses, as a result of the temperature inside the nasal cavity during the operations. To deal with this, surgeons have to use demisting solution repeatedly to get a clear field, which again involves additional time, as well as the expense of the demisting solution.

In addition, control of suction and power of suction can be a problem during nasal surgeries. Existing suction instruments used in nasal surgeries provide no control to enable increased or decreased control of suction. They are usually connected directly to an external suction device and the only way to control the strength of the suction is by increasing or decreasing the power of the external suction device. There is no control within the suction instrument, at the site of the operation. This uncontrolled suction power can cause collapse of the nasal cavity in children and infants and can cause problems in fine cosmetic surgery procedures such as rhinoplasty.

In the case of multiple facial and head injuries, it is currently very difficult immediately to protect the airway from post nasal bleeding without intubating the patient.

Devices have been disclosed previously which act to provide a combination of suction with a means of occluding the airway so that fluids cannot drain into the airway. Some devices include use of an inflatable balloon portion, for example U.S. Pat. No. 6,027,478, WO2005/117755. However, the prior devices only enable suction and occlusion to occur in different locations, i.e., suction takes place at a location either in front of or behind the inflated balloon causing the occlusion. This not only potentially reduces the efficacy of suction (depending on where the fluid is coming from) but also increases the overall volume of apparatus which is present in the operative space or the surrounding areas. In addition, the devices do not address the issue of how to apply and maintain medicaments at a required location within the nasal, nasopharangeal and oropharangeal spaces.

### SUMMARY OF INVENTION

According to a first aspect of the invention, there is provided a suction device comprising a funnel end connected to a suction tube connectable to a vacuum device, the funnel end comprising a reversibly inflatable portion. The device is preferably a nasopharangeal device suitable for insertion through a mouth of a patient, the funnel end being formed so as to be sealably locatable in or around the nasopharangeal opening of the patient.

The patient is preferably a human being, although the device may also be suitable for use with other mammals such as cats, dogs and horses, by way of non-limiting example. The term “nasopharangeal opening” indicates the opening between the nasopharangeal space and the oropharangeal space, i.e., the opening located at the back of the throat which links the nasal cavity to the pharynx. The term “sealably locatable” indicates that the open mouth of the funnel end, when positioned by a user in the correct location, forms a reversible seal (which may be a perfect or imperfect seal) in or around the nasopharangeal opening such that fluids in the nasal cavity are substantially prevented from moving through the pharynx into, for example, the airway of the patient. The seal may be formed by contact of the mouth of the funnel end with the tissue forming and/or surrounding the nasopharangeal opening. Therefore, the open mouth of the funnel is of a size and shape which facilitates the formation of such a seal.

The term “funnel end” is intended as a general term indicating a structure having an open mouth connected to an exit opening, with the structure of the funnel end directing any fluid entering the open mouth towards the exit opening. Therefore, the funnel end need not be a conical funnel shape but may be, for example, formed in the shape of a cup or trough. The funnel end might, for example, be formed in a shape similar to a tobacco pipe, with an elongate portion and a wider mouth portion. The mouth of the funnel end may be circular but may also be elliptical or be a straight-sided geometric shape (e.g., a rectangle) with curved corners. Most suitably, the mouth of the funnel end may be formed to resemble the shape of the nasopharangeal opening of a patient, for example, a human being, such that, when the mouth of the funnel is positioned over or within the nasopharangeal opening, a seal is more readily formed. When the device is not a nasopharangeal device, the funnel end may be formed in any suitable shape for use in a different part of the body. The open mouth of the funnel end may be wider than or the same size as the suction tube, i.e., it may have an external dimension (such a diameter) greater than or equal to the equivalent dimension of the suction tube.

Advantageously, the device according to the invention enables a surgeon carrying out nose surgery to close off the nasopharangeal opening such that fluids derived from the nasal cavity cannot drain into the patient's airway. Simultaneously, suction may be applied by the vacuum device to forcibly extract fluid through the mouth of the funnel end away from the device, through the suction tube. The vacuum device (or aspiration device) may be of the type typically found in surgical theatres, or may be a simple device such as a syringe. The speed and volume of suction may be controlled, for example, by any suitable system which enables controllable alteration of the strength of the suction applied though the mouth of the funnel end. Such a system may be incorporated into the device to form an integral part of the device.

For example, the device may comprise at least one tube aperture formed in a side wall of the suction tube and a collar encircling the tube and moveable to reversibly cover the aperture. At least one collar aperture or hole, each of which may differ in size to the other collar apertures or holes, may be formed in a wall of the collar and the collar may be moveable to reversibly locate at least one collar aperture in relation to at least one tube aperture such that the tube aperture is uncovered. Where the suction tube aperture(s) are uncovered, for example because the one or more collar holes are positioned over the suction tube aperture(s), the strength of suction provided at the funnel end mouth will be reduced compared to the strength when the suction tube aperture(s) are covered by the material of the collar.

Such control systems enable control of misting of endoscopic lenses by the action of sucking air through the operative space in a controlled manner. This also facilitates clearing of smoke in laser surgeries, cauterisation and diathermy procedures. Therefore, good visualisation of the operative field is possible. The ability to control the suction also prevents excessive suction and possible collapse of the operative field. The suction can readily be controlled by an assistant who is required to make less or no use of demisting solutions, saving operative time.

The fact that the funnel end comprises a reversibly inflatable portion allows the user to control the pressure at which the mouth of the funnel end is applied to the tissue forming and/or surrounding the nasopharangeal opening, thereby controlling the effectiveness of the seal and also minimising damage to the tissue which might be caused by the applied pressure. The reversibly inflatable portion may be attached to any inflation/deflation device, most simply a device such as a syringe which may be utilised to increase or decrease the amount of air inside the inflatable portion. The inflation/deflation device may be attached via a connector such as is commonly used with anaesthetic endotracheal tubes, incorporating an inflatable bulb the pressure of which corresponds to and/or correlates with the pressure in the reversibly inflatable portion of the funnel end. This enables the user to assess and control the extent of inflation of the funnel end inflatable portion. Alternatively or additionally, such an inflation/deflation device and/or connector may also comprise a sensor to detect the level of pressure within the inflatable portion and the inflation/deflation device may be partially or fully automated, so that the inflatable portion may be maintained at a pre-determined pressure during the procedure in which the device is used. Air, or any other suitable fluid, may be used to inflate the inflatable portion.

The funnel end may wholly be formed from the inflatable portion, such that the material forming the funnel end is flaccid when the inflatable portion is uninflated and forms a three-dimensional shape only when the inflatable portion is inflated. However, in a preferred embodiment, the funnel end is formed from a rigid or semi-rigid deformable material which maintains a funnel shape at all times. The funnel end may then comprise an inflatable sheath formed across at least a portion of the external surface of the funnel end. Alternatively or additionally, the reversibly inflatable portion of the funnel end may comprise an inflatable cuff formed around the mouth of the funnel end. The sheath enables all or a portion of the funnel end to be increased in size by inflation of the sheath, facilitating the formation of a seal in and/or around the nasopharangeal opening. The cuff may, alone or in combination with a sheath on the exterior surface of the funnel end, further improve formation of the required seal by enabling an increased pressure in the inflatable portion around the mouth of the funnel end.

In a preferred embodiment, the device according to the invention may further comprise an obturator insertable into the suction tube. An obturator may be, for example, a length of material which is at least as rigid, or more rigid than, the material forming the suction tube, such that the obturator may be inserted into the suction tube like a piston in a cylinder. This enables the insertion of medicaments and/or packing materials into the device which may exit the device via the mouth of the funnel end and be held against the tissue of the patient by the insertion of the obturator, with the structure of the funnel mouth additionally assisting with maintaining the required positioning of the medicament and/or packing material. In a major advantage of this aspect of the invention, this enables simultaneous sealing of the nasopharangeal opening with application of medicaments and/or packing materials, so that the airway of the patient is not left exposed to nasal-derived fluid whilst such medicaments or materials are positioned.

The obturator may also assist with initial insertion of the device and subsequent adjustments in position, since the presence of the obturator increases the overall rigidity of the device.

The suction tube of the device may comprise a fluid removal tube or opening and an access tube or opening. This is especially in the case where the device comprises an obturator, so that the obturator can be inserted into the device via the access tube, whilst maintaining suction of unwanted fluids from the nasal cavity via the fluid removal tube, connected to a vacuum device. This provides the particular advantage that suction can be maintained simultaneously with the application of medicaments and/or packing materials, although the amount of suction may be reduced by the presence of the obturator. However, the access tube might also be used as an access point for endoscopic surgical devices.

The device may be for use during a surgical method, for example in a method of protecting an airway of a patient from nasal-derived liquid or in a method of applying medicament and/or packing to a nasopharynx, oropharynx or nasal cavity of a patient.

Features of the device may be altered depending on the patient with which the device is to be used. For example, a device intended for paediatric use would have reduced dimensions compared to a device intended for use with adult patients. A device for use with babies would be even smaller in size. The device might also be used with non-human patients such as dogs, cats or horses, for example, in which case the shape of the funnel and/or the mouth of the funnel, as well as the shapes of other parts of the device, might require alteration according to the anatomy of the animal. The essential feature of the mouth of the funnel end is that it is capable of forming a seal in or around the nasopharangeal opening of the patient by making sealable contact with the tissue forming and/or surrounding the nasopharangeal opening. The formation of the seal is facilitated by the inclusion of the inflatable portion of the funnel end, which may form, for example, a sheath on the exterior surface of the funnel end and/or a cuff around the mouth of the funnel end, as has been outlined above and as is exemplified below.

The device may include external markings on the body of the device, to assist the user in determining the extent of insertion into the patient and/or the orientation of the funnel mouth to the nasopharangeal opening.

The device may be formed from any material suitable for use in surgical procedures according to the requirements of elasticity, rigidity and strength required for each part of the device. For example, it may be desirable for the suction tube to be fairly rigid, as well as the fluid removal and access tubes, where present. Similarly, it is desirable for the obturator to be rigid so as to enable insertion into the device and to facilitate the positioning of any medicaments and/or packing materials. Suitable materials may be, therefore, metals and/or hard plastics materials, which may be sterilisable such that the device, or these portions of the device, are reusable. In contrast, it might be helpful for the material forming the funnel end to be deformable, for example to facilitate insertion of the device through the mouth and into the region adjacent the nasopharangeal space. Therefore, a semi-rigid but deformable plastics material might be utilised for this part of the device, which might be separable from the rest of the device and which may be disposable even if the rest of the device is reusable. However, if a sterilisable material is used, the funnel end portion of the device may also be reused. Finally, the inflatable portion of the funnel end may be a material which is elastic and expandable, so that it may expand in size when air (or another fluid) is introduced into the inflatable portion using the inflation/deflation device. For example, a silicone polymer material may be used.

In emergencies, the device can be used to protect the airway from posterior nasal bleeding immediately, without the need of intubation. Combined with anterior nasal packing with the obturator in place, this device immediately controls postnasal bleeding and avoids the need of intubation or postnasal packing during emergencies.

According to a second aspect of the invention there is provided a method of protecting an airway of a patient from nasal-derived liquid comprising inserting a device according to the first aspect of the invention through the oral cavity of a patient, positioning the open end of the funnel end adjacent the nasopharyangeal space of the patient and inflating the inflatable portion of the funnel. Positioning the funnel end opening adjacent the nasopharyangeal space comprises locating the mouth of the funnel end such that it can locate within or cover or substantially cover the nasopharyangeal opening. The method may then further comprise the step of inflating the inflatable portion of the funnel end such that a seal is reversibly formed between the mouth of the funnel end and the tissue surrounding the nasopharyangeal opening.

According to a related aspect of the invention, there is provided a method of applying medicament and/or packing to a nasopharynx, oropharynx or nasal cavity of a patient comprising inserting a device according to the first aspect of the invention (including an obturator) through the oral cavity of a patient, positioning the open end of the funnel end adjacent the nasopharyangeal space of the patient and inflating the inflatable portion of the funnel end, applying the medicament and/or packing to a distal end of the obturator and inserting the obturator into the suction tube of the device. The distal end of the obturator is the end which is first inserted into the device, in use. Positioning the funnel end opening adjacent the nasopharyangeal space comprises locating the mouth of the funnel end such that it can locate within or cover or substantially cover the nasopharyangeal opening. The method may then further comprise the step of inflating the inflatable portion of the funnel end such that a seal is reversibly formed between the mouth of the funnel end and the tissue surrounding the nasopharyangeal opening. The insertion of the obturator into the suction tube of the device, for example via the access tube where present, enables the medicament and/or packing to be positioned towards the mouth of the funnel end, in most situations exiting the mouth of the funnel to contact tissue located within the boundary formed by the funnel end mouth.

The methods of the second aspect of the invention may be included within surgical procedures which may be carried out on a human being or on an animal such as a dog, cat or horse, by way of non-limiting example.

According to a third aspect of the invention there is provided a surgical kit comprising a device according to the first aspect of the invention. The kit may further comprise a vacuum device and/or an inflation/deflation device for use with the device as outlined above, either of which may be a syringe. The kit may further comprise packing material such as gauze or wadding, or medicaments which may be separate or applied to the packing material, any of which may be positioned at a required location in the patient by use of a device according to the invention comprising an obturator. The kit may be for use in one or more methods according to the second aspect of the invention.

Throughout the description and claims of this specification, the words “comprise” and “contain” and variations of the words, for example “comprising” and “comprises”, mean “including but not limited to” and do not exclude other additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Other features of the present invention will become apparent from the following examples. Generally speaking, the invention extends to any novel one, or any novel combination, of the features disclosed in this specification (including the accompanying claims and drawings). Thus, features, integers and characteristics, described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The present invention has been disclosed herein particularly in the context of a device for use in the nasopharangeal region of a patient. However, the skilled person will readily be able to make the routine alterations which would be required to adapt the device for use in other parts of the body where combined suction and passage occlusion would be useful, particularly where positioning and maintenance of a medication and/or packing material at a particular location would be advantageous. Non-limiting examples include the uterus, the cervix, the bowel, the neck, the eye and eye orbit, the chest, the bladder and the abdomen.

### BRIEF DESCRIPTION OF FIGURES

Embodiments of the invention will now be shown, by way of example only, with reference to the accompanying Figures in which:

FIG. 1 shows a side view of a device according to the invention;

FIG. 2 shows a view of the device from above;

FIG. 3 shows an alternative embodiment of the device;

FIG. 4 shows the location of a device according to the invention when in use with a patient;

FIG. 5 shows embodiments of the structure of an inflatable cuff of a device;

FIG. 6 shows embodiments of a suction control collar encircling a suction tube; and

FIG. 7 shows a side view of a device according to the invention incorporating a suction control collar shown in FIG. 6.

### EXAMPLES

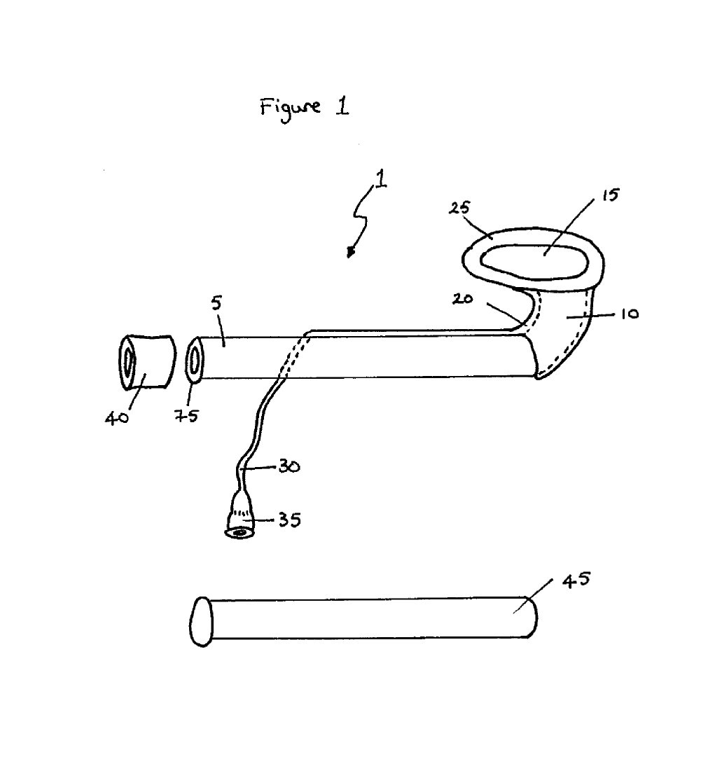

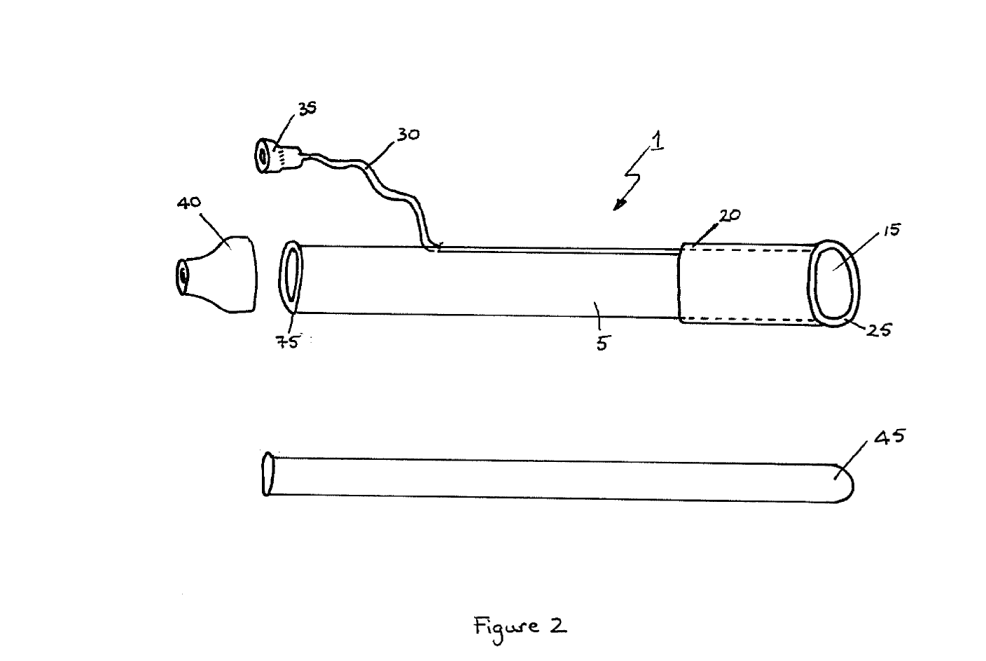

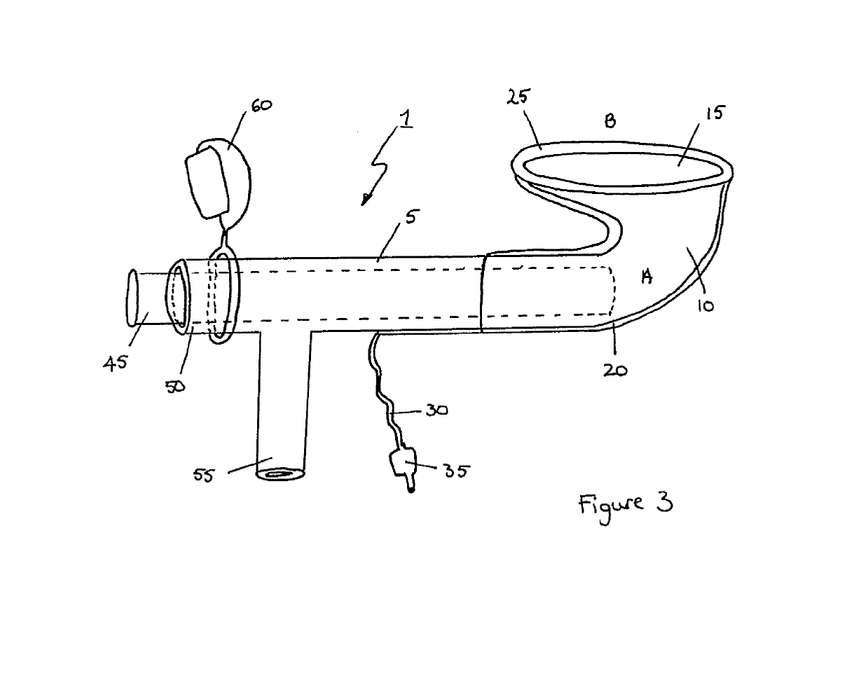

The nasopharangeal device is shown in FIGS. 1 and 2, with a slightly different version shown in FIG. 3. The device 1 includes a tube section 5 and a funnel section 10. The funnel has a mouth 15 which is shaped so as to be able to make contact with the tissue in and around the nasopharangeal opening. The outside of the funnel 10 is surrounded by a sheath 20 which includes a cuff section 25 encircling the mouth 15 of the funnel 10. The sheath 20 and cuff section 25 are inflatable and/or deflatable via an inflation tube 30 which can be connected to an inflation/deflation device (such as a syringe) via connector 35. The end 75 of the tube section 5 can be connected to a suction device via connector 40, to facilitate suction of fluids through the device via the funnel mouth.

FIGS. 1 and 2 also show an obturator 45 outside the device, the obturator 45 being a solid cylinder of, for example, a plastics material. FIG. 3 shows the obturator 45 in place within the device 1, having been inserted into the tube section 5 via an access tube 50. The embodiment of the device shown in FIG. 3 also includes a separate fluid removal tube 55, enabling suction through the device to continue even when the obturator 45 is inserted into the device. The obturator enables medicaments and/or packing materials to be placed either within the device in the funnel in the area shown as “A” (for example if a slow release medication is to be used), or outside the mouth of the funnel in the area shown as “B”, for example, against the tissue adjacent the funnel mouth. This embodiment of the device also includes a cap 60 which can be used to close off the access tube 50 when not in use for access of an obturator or surgical instrument. In addition, the inflation/deflation device connector 35 is of a type commonly seen in anaesthetic endotracheal tubes, incorporating a bulb the pressure of which correlates with the pressure in the cuff 25. This enables the user to assess and control the degree of inflation of the cuff.

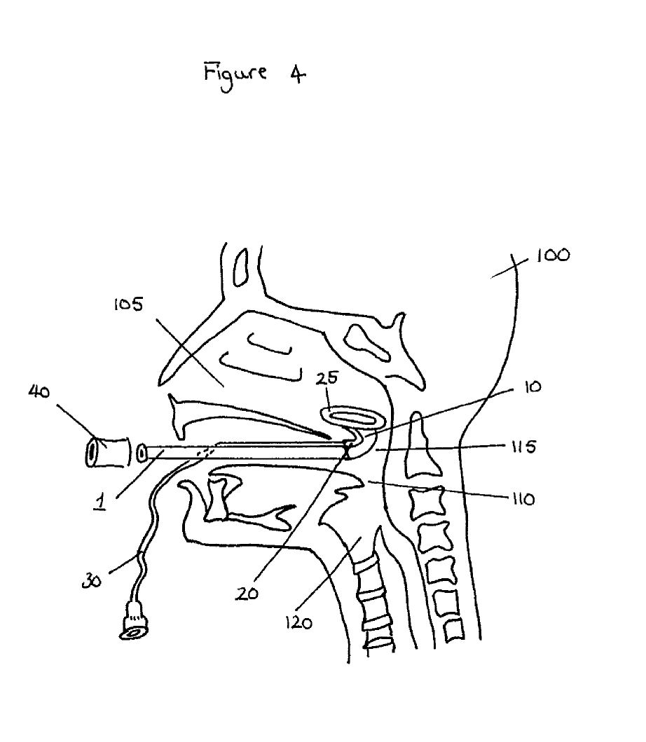

FIG. 4 shows a section through a patient's head 100 showing approximately how the device 1, in use, is positioned within a patient. The nasal cavity 105 of the patient is linked to the pharynx 110 and then airway 120 by the nasopharangeal opening 115. The funnel 10 of the device 1 is positioned in the nasopharangeal opening 115 and the sheath 20 and cuff 25 can be inflated via the inflation tube 30 so as to form a seal between the funnel 10 and the nasopharangeal opening 115. Therefore, fluids formed in the nasal cavity 105 are prevented from moving into the pharynx 110 and subsequently into the airway 120.

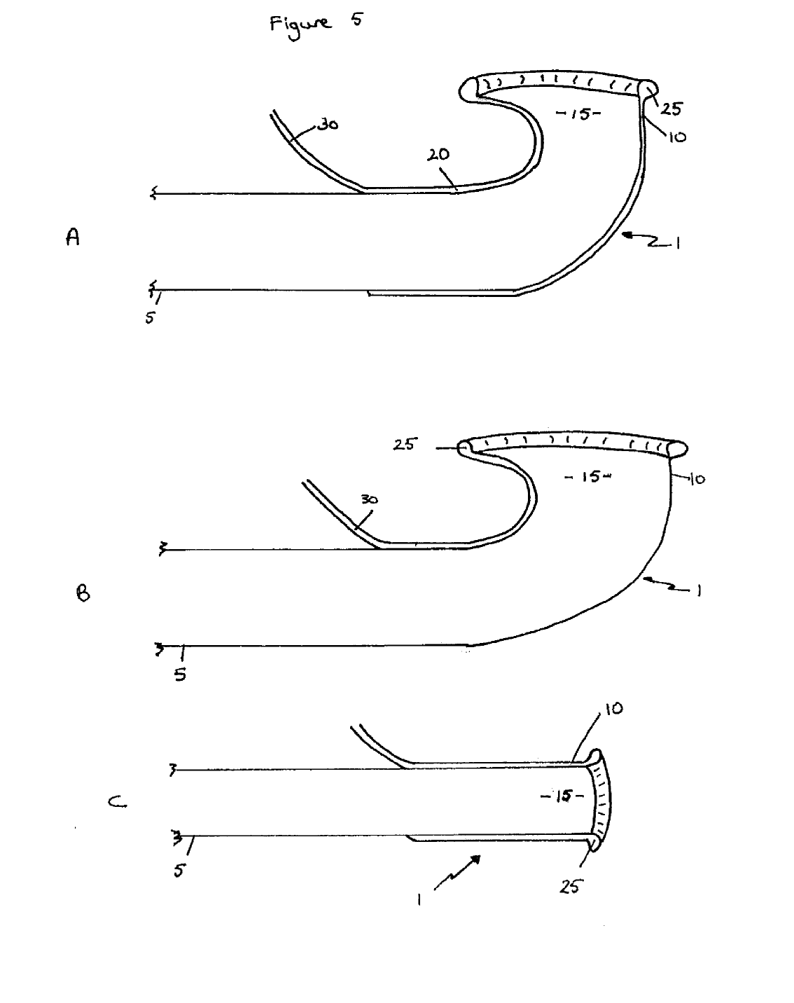

FIG. 5 shows cross-sections of various embodiments of the funnel mouth 15 of the device 1 and the inflatable sheath 20 and cuff 25. FIG. 5A shows a sheath 20 which surrounds the funnel end of the device 1. The interior of the sheath 20 is connected to the interior of the cuff section 25 which encircles the mouth 15 of the funnel, so that both the sheath 20 and the cuff 25 are simultaneously inflatable or deflatable via the tube 30. In contrast, FIG. 5B shows an inflatable cuff 25 which is directly inflatable or deflatable via the tube 30, with no sheath portion surrounding the rest of the funnel end of the device. FIG. 5C shows a device having a differently shaped funnel section, for example when intended for use in a non-nasopharyngeal setting. As in FIG. 5A, in this embodiment there is a sheath 20 and cuff 25, both inflatable via the tube 30, though this type of funnel end may also only comprise an inflatable cuff in the absence of a sheath, as shown in FIG. 5B.

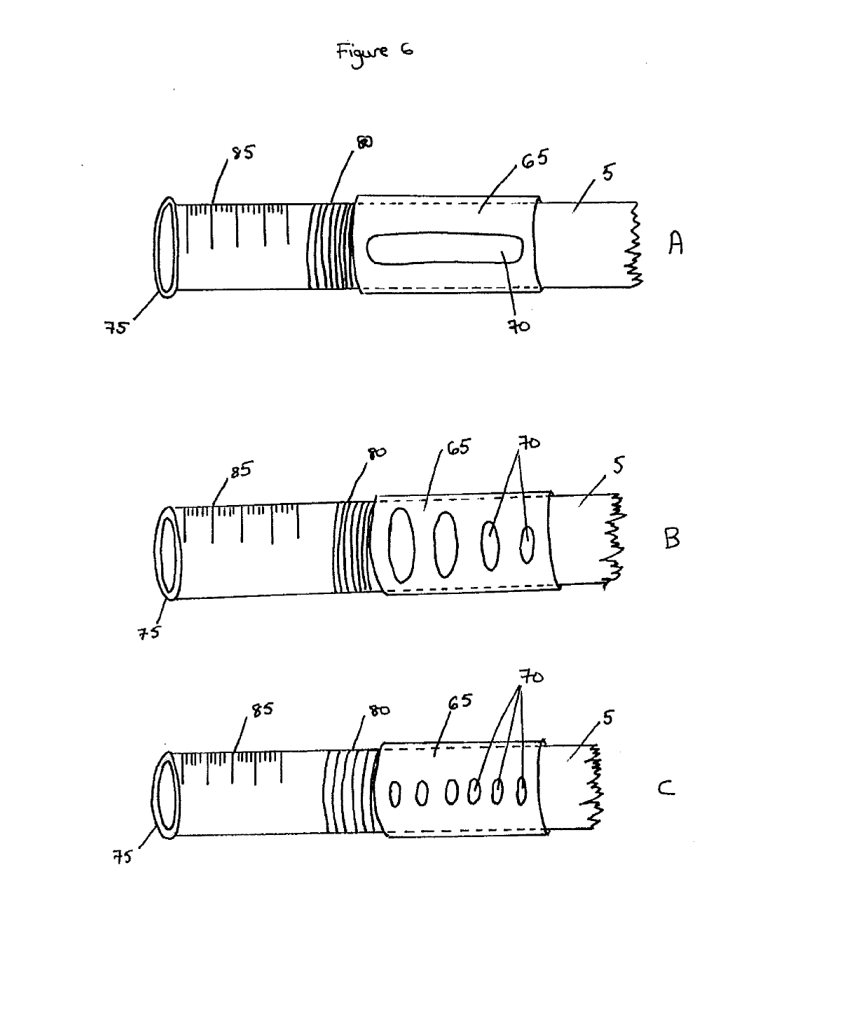

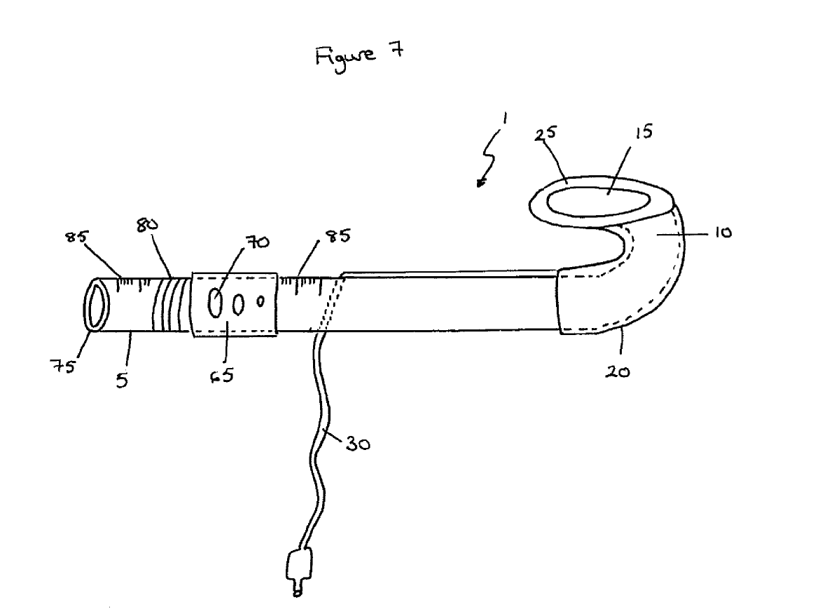

FIG. 6 shows embodiments of a system for controlling the strength of suction through the suction tube 5. The end 75 of the tube 5 is connectable to an external suction device. Control of suction may be facilitated by a collar 65 which encircles the exterior of the tube 5, lateral movement along the length of the tube being controllable by use of screw threads between the collar and the tube, those formed on the tube being indicated as 80. This enables rotation of the collar 65 around the tube 5, with the screw thread 80 operating to cause lateral movement of the collar along the length of the tube, towards or away from the end 75. The collar sits over a hole (not shown) formed in the tube 5 and the collar itself comprises one or more holes 70 which, when positioned over the hole formed in the tube 5, alters the strength of the suction applied through the tube 5 to the funnel opening 15 (not shown in FIG. 6). Markings 85 may be provided on the body of the suction tube 5, to assist the user in determining the appropriate position of the collar 65 and, therefore, the strength of suction provided. FIG. 7 shows a side view of a device incorporating a collar 65 as described.

1. A nasopharangeal suction device for insertion through a mouth of a patient, comprising a funnel end connected to a suction tube connectable to a vacuum device, the funnel end being formed so as to be sealably locatable in or around the nasopharangeal opening of the patient and comprising a reversibly inflatable portion.

2. The device according to claim 1, wherein the reversibly inflatable portion of the funnel comprises an inflatable sheath formed across at least a portion of the external surface of the funnel end.

3. The device according to claim 1, wherein the reversibly inflatable portion of the funnel end comprises an inflatable cuff formed around the mouth of the funnel end.

4. The device according to claim 1, wherein the reversibly inflatable portion is connected to an inflation device.

5. The device according to claim 1, comprising at least one tube aperture formed in a side wall of the suction tube and a collar encircling the tube and moveable to reversibly cover the aperture.

6. The device according to claim 5, wherein at least one collar aperture is formed in the collar and the collar is moveable to reversibly locate at least one collar aperture in relation to at least one tube aperture such that the tube aperture is uncovered.

7. The device according to claim 1, further comprising an obturator insertable into the suction tube.

8. The device according to claim 1, wherein the suction tube comprises a first suction opening and a second access opening.

9. A method of protecting an airway of a patient from nasal-derived liquid, comprising inserting a device according to claim 1, into the oral cavity of a patient, positioning the open end of the funnel end adjacent the nasopharyangeal space of the patient and inflating the inflatable portion of the funnel end.

10. A method of applying medicament and/or packing to a nasopharynx, oropharynx or nasal cavity of a patient, comprising inserting a device according to claim 1, comprising an obturator insertable into the suction tube, into the oral cavity of a patient, positioning the open end of the funnel end adjacent the nasopharyangeal space of the patient and inflating the inflatable portion of the funnel end, applying the medicament and/or packing to a distal end of the obturator and inserting the obturator into the suction tube of the device.

11.-14. (canceled)

15. A surgical kit comprising a device according to claim 1.

16. (canceled)
